(19) United States
(12) Patent Application Publication
(10) Pub. No.: US 2021/0363087 A1
(43) Pub. Date: Nov. 25, 2021

(54) Chemical Conversion of Carbon Dioxide and Gaseous Hydrocarbons to Solvents

(71) Applicant: Melvin Keith Carter, Lincoln, CA (US)

(72) Inventor: Melvin Keith Carter, Lincoln, CA (US)

(21) Appl. No.: 16/878,836

(22) Filed: May 20, 2020

(51) Int. Cl. C07C 45/27; C07C 2/86; C07C 29/03

(52) U.S. Cl. CPC C07C 45/27; C07C 9/15; C07C 29/03; C07C 2/862

(57) ABSTRACT

Catalytic chemical conversion of carbon dioxide gas combined with a hydrocarbon gas, comprising natural gas, methane, ethane, propane, butane or pentane, over a transition metal oxide, produces organic solvent products. The process converts oxidized carbon compounds to valued organic products and can reduce or eliminate the carbon footprint of industrial electric power generation industry. Catalytic processes are taught for chemical conversion of oxides of carbon, principally carbon dioxide (formed by combustion of hydrocarbons), to solvent compounds comprising acetone, butanol, pentane and related organic products. The catalysts are transition metal oxides, selected from the group comprising manganese, iron and cobalt or combinations thereof.

### REFERENCES CITED

### U.S. Patent Documents

| Patent No. | Issue Date | Author | Comments |
|---|---|---|---|
| 8,980,961 | Mar. 17, 2015 | G A Olah, | Chemical conversion of carbon dioxide and natural gas to synthesis |
| | | G K S Prakash | gas then methanol over a mixed oxide catalyst at 800° C. to 1100° C. |
| 8,609,385 | Dec. 17, 2013 | S Anderson | Using genetically engineered photosynthetic microorganisms to |
| | | | convert carbon dioxide into isoprene (a biological process). |
| 8,440,729 | May 14, 2013 | G A Olah, | Chemical conversion of carbon dioxide and natural gas to synthesis |
| | | G K S Prakash | gas then methanol over a Ni—V oxide catalyst at 800° C. to 1100° C. |
| 8,133,926 | Mar. 13, 2012 | G A Olah, | Chemical conversion of carbon dioxide and natural gas to synthesis |
| | | G K S Prakash | gas then methanol to dimethyl ether over a catalyst of polymeric |
| | | | perfluoroalkanesulfonic acid. |
| 7,973,199 | Dec. 17, 2013 | S Anderson | Production of isoprene from carbon dioxide using photosynthetic |
| | | | microorganisms. |
| 7,282,613 | Oct. 16, 2007 | J R Black, | A process for producing phenol and MEK from organic reactants. |
| | | J Yang, | |
| | | J L Buechele | |
| 4,861,923 | Aug. 29, 1989 | G Olah | Hydration of propylene to isopropanol on superacidic |
| | | | perfluorinated sulfonic acid catalysts |

### BACKGROUND

### Field of Invention

Approximately 63 percent of electric power is generated by means of combustion of coal and/or hydrocarbon fuels producing large volumes of carbon dioxide gas. The world's atmosphere and ocean waters have become laden with abundant carbon dioxide gas believed to be the cause of global warming and acidification of ocean waters. Some recent power plants have been designed with combustion gas scrubbers to remove and isolate carbon dioxide. By converting this gas to useful products the tendency toward increasing concentrations of carbon dioxide in the atmosphere could be short circuited from the environment to commerce.

The National Oceanic and Atmospheric Administration (NOAA) recorded carbon dioxide concentrations in the atmosphere at Mauna Loa, Hi. of 317 ppm in 1960 that have risen to over 410 ppm in 2020. NASA's Orbital Carbon Observatory indicated some 720 billion tons of carbon dioxide gas have been measured in Earth's atmosphere. The Earth's oceans contain 37,400 billion tons of carbon dioxide and continue to act as concentration buffers for Earth's atmosphere. Humans generate 6 billion tons/year, herd animals contribute 3.1 billion tons/year while combusted coal and hydrocarbon fuels contribute another 19.9 billion tons/year. In addition, carbon dioxide comprises approximately forty five percent of biogas emanating from landfill.

Continued irresponsible dumping of carbon dioxide into Earth's atmosphere could lead to extinction of life on this planet as we know it. Captured carbon dioxide gas can be reacted with slacked lime to produce stabilizing lime stone, a relatively expensive process. Alternatively, it may be converted to useful industrial products. One way to begin to address this problem is to capture the carbon dioxide from combustion gases and to convert it into useable solvents as opposed to pumping it into deep underground wells from which seepage back into the atmosphere is possible. This application of Chemical Conversion of Carbon Dioxide and Gaseous Hydrocarbons to Solvents teaches the chemical process.

This catalytic chemical process converts oxidized carbon compounds to valued organic compounds and can reduce or eliminate the carbon footprint of industrial processes. A catalytic process is taught for chemical conversion of carbon dioxide combined with hydrocarbon gases comprising natural gas, methane, ethane, propane, butane or pentane to valued organic solvents comprising acetone, methyl ethyl ketone, pentanone, hexanone, heptanone, octanone, butanol, pentanol, hexanol, heptanol, octanol, pentane, hexane, heptane, octane and higher molecular weight hydrocarbon compounds.

The catalyst compounds of transition metals comprise oxides of manganese, iron, cobalt or combinations thereof. These conversion reactions are conducted at elevated temperatures in the range of 200° C. to 500° C. and pressures of 1 to 10 atmospheres.

### Description of Prior Art

U.S. Pat. No. 8,980,961 issued Mar. 17, 2015, reduced to practice by G. A. Olah and G. K. S. Prakash report chemical conversion of carbon dioxide and natural gas to synthesis gas followed by chemical conversion to methanol over a mixed catalyst at 800° C. to 1,100° C.

U.S. Pat. No. 8,609,385 issued Dec. 17, 2013, reduced to practice by S. Anderson teach production of isoprene by the direct conversion of atmospheric carbon dioxide using metabolically engineered/genetically engineered photosynthetic microorganisms (a biological process). Their invention also relates to genetically engineered photosynthetic microorganisms, such as cyanobacteria, that are capable of producing isoprene from carbon dioxide.

U.S. Pat. No. 8,440,729 issued May 14, 2013, reduced to practice by G. A. Olah and G. K. S. Prakash disclose chemical conversion of carbon dioxide and natural gas to synthesis gas followed by chemical conversion to methanol over a mixed nickel vanadium catalyst at 800° C. to 1,100° C.

U.S. Pat. No. 8,133,926 issued Mar. 13, 2012, reduced to practice by G. A. Olah and G. K. S. Prakash, disclose chemical conversion of carbon dioxide and natural gas to synthesis gas followed by chemical conversion to methanol which is subsequently converted to dimethyl ether. The catalyst is a metal oxide or a polymeric perfluoroalkane sulfonic acid resin.

U.S. Pat. No. 7,973,199 issued Dec. 17, 2013, reduced to practice by S. Anderson, teaches production of isoprene from carbon dioxide using photosynthetic microorganisms.

U.S. Pat. No. 7,282,613 awarded Oct. 16, 2007, reduced to practice by J. R. Black, J. Yang and J. L. Buechele of Shell Oil Company teaches a process for producing a combination of products selected from the group consisting of (a) phenol and methyl ethyl ketone (MEK) or (b) phenol, acetone, and MEK, said process comprising feeding one or more alkylbenzenes selected from the group consisting of (a) a content of s-butylbenzene, and (b) a combination of s-butylbenzene and cumene cleaving product hydroperoxides to produce products comprising a combination selected from the group consisting of (a) phenol and MEK, and (b) phenol, acetone, and MEK. This is a chemical process that used no carbon dioxide.

U.S. Pat. No. 4,861,923 awarded Aug. 29, 1989, reduced to practice by G. A. Olah discloses propene reacts with water in a strong sulfuric acid solution at 20 to 25 atmospheres pressure producing isopropyl alcohol at ambient temperature. This product can be dehydrated at 3 to 4 atmospheres pressure and 400° C. to 500° C. exposed to a copper oxide catalyst to form acetone. This two-step process can produce other products, however no carbon dioxide was consumed.

None of these patented processes teach direct chemical reaction of carbon dioxide with hydrocarbon gases for the formation of acetone, butanol, pentane or related solvent products.

### SUMMARY OF THE INVENTION

This invention describes a chemical process, using selected transition metal oxide catalysts, for conversion of carbon dioxide mixed with gaseous hydrocarbons to solvents. The process is direct in that carbon dioxide mixed with a gaseous hydrocarbon medium comprising natural gas, methane, ethane, propane, butane or pentane in the presence of a transition metal oxide catalyst, selected from the group manganese oxide, iron oxide and cobalt oxide, reacts at 200° C. to 500° C. and 1 atmosphere to 10 atmospheres pressure produces solvents comprising acetone, butanol, and pentane directly.

It is an object of this invention, therefore, to provide transition metal oxide catalysts, selected from the group manganese oxide, iron oxide or cobalt oxide, for chemical conversion of carbon dioxide combined with gaseous hydrocarbons, comprising natural gas, methane, ethane, propane, butane or pentane, to solvents. It is another object of this invention to provide catalytic processes for chemical conversion of carbon dioxide combined with gaseous hydrocarbons, comprising natural gas, methane, ethane, propane, butane or pentane, to useful organic solvents. Other objects of this invention will be apparent from the detailed description thereof that follows, and from the claims.

### DETAILED DESCRIPTION OF THE INVENTION

A process is disclosed for catalytic chemical conversion of carbon dioxide mixed with gaseous hydrocarbon compounds, comprising natural gas, methane, ethane, propane, butane or pentane, to solvents using selected members of a family of transition metal catalysts, comprising manganese oxide, iron oxide, cobalt oxide or combinations thereof. Solvent products were formed from carbon dioxide and gaseous hydrocarbons using two different chemical conversion processes to prove product formation. Solvents formed from a pressure reactor were isolated for testing and products produced in a flow through reactor were confirmed by Fourier Transform Infrared spectroscopy (FTIR) as disclosed in the Chemical Conversions that follow.

The process for catalytic reaction of carbon dioxide with gaseous hydrocarbons comprising natural gas, methane, ethane, propane, butane or pentane is demonstrated in production of solvents comprising acetone, butanone, pentanone, hexanone, heptanone, octanone, nonanone, decanone and undecanone, butanol, pentanol, hexanol, heptanol, octanol, nonanol, decanol, undecanol, dodecanol, tridecanol, tetradecanol, pentadecanol, hexadecanol, pentane, hexane, heptane, octane, nonane, decane, undecane, dodecane, tridecane, tetradecane, pentadecane, hexadecane, heptadecane, octadecane, nonadecane, icosane and heneicosane. The chemistry reveals a reaction of one of the oxygen atoms of carbon dioxide with a hydrogen atom donated by each of two hydrocarbon molecules to form, for example, acetone solvent and the byproduct water. It also reveals carbon dioxide reacting with each of three hydrocarbon molecules to form, for example, butanol and carbon dioxide reacting with each of four hydrocarbon molecules to form, for example, pentane and the byproduct water.

The process is based on transition metal oxide catalysis to facilitate conversion of carbon dioxide mixed with gaseous hydrocarbon compounds at temperatures, in the range of 200° C. to 500° C. and reaction pressures in the range of 1 atmosphere to 10 atmospheres, to produce solvents.

### Chemical Conversions

A first set of catalytic chemical conversions was conducted using a 300 mL high-pressure reactor. Products were identified by physical properties and boiling point. The reactor was fit with two valves to facilitate gas flow in and gas flow out. It was closed and sealed by means of a bolt assembly. Approximately 5 grams of anhydrous magnesium chloride (included to assure residual water was absorbed) was combined with 7.5 grams of ⅛″ long cylindrical silica-alumina support coated with 2 percent by weight cobalt oxide. The reactor was heated to 310° C. and flushed with bone dry carbon dioxide gas. It was cooled, pressurized with 5 psig bone dry carbon dioxide and the pressure increased to 47 psig with dried natural gas (95% methane). The reactor was heated to 450° C. for an hour and fifteen minutes as the pressure declined. The reactor was cooled to ambient temperature and opened. Solvent liquid, 0.815 gram, was isolated from the remaining solid by filtration. The solvent had a pungent odor and a boiling point of 56° C. identified as acetone.

A second reaction was conducted by loading 5 grams of anhydrous magnesium chloride was combined with 7.5 grams of ⅛″ long cylindrical silica-alumina support coated with 2 percent by weight manganese oxide. The reactor was heated to 310° C. and flushed with bone dry carbon dioxide gas. It was cooled, pressurized with 23.5 psig bone dry carbon dioxide and the pressure increased to 100 psig with dried propane. The reactor was heated to 450° C. for an hour and one half as the pressure declined. The reactor was cooled to ambient temperature and opened. Solvent liquid, 0.865 gram, was isolated from the remaining solid by filtration. The solvent had a light odor of bananas and a boiling point of 151° C. identified as heptanone.

A second set of catalytic chemical conversions was conducted using ½ inch diameter stainless steel tube flow through reactor configured in sections. The front end consisted of a carbon dioxide flow control valve, a hydrocarbon gas flow control valve and an inlet for admission of gaseous reactants. Next section was a 3 inch drier mixing tube filled with molecular sieves 4 A. The final section of the reactor was terminated by a right angle reducer to a ¼ inch diameter tube connected to a 10 mL condensing finger immersed in a cold bath (either dry ice/acetone or ice/water baths were employed) and flow gases were exhausted. Each section was heated using electrical heating tape connected to temperature controllers with section (zone) temperatures monitored using thermal couples. Resulting products were recovered from a cold trap and identified by means of FTIR spectral band absorptions.

### Example A—Acetone

The reactor zone was heated to 280° C. as carbon dioxide and natural gas were supplied at 1.4 atmospheres (20 pounds per square inch) pressure with a total flow rate of 214 mL/minute. Carbon dioxide was set to 70 mL/minute while natural gas was set to 144 mL/minute. Gases were allowed to flow for 30 minutes. A colorless liquid, weight was 6.64 grams, was collected in the cold trap immersed in an ice water bath.

The liquid was dried over anhydrous sodium sulfate then filtered to collect 4.8 grams of liquid product. An FTIR spectrum of the liquid produced useful spectral information. Relatively intense absorption bands were observed at 2966, 2937 and 2875 wave numbers with weaker bands at 1467, 1379, 1276, 905, 886, 798 and 754 wave numbers. In addition, a relatively intense band was observed at 1715 wave numbers identified with ketones with a weak band at 1199 wave numbers indicating the presence of an alcohol. This was identified as acetone, as produced in the previous section, and a lesser amount of butyl alcohol.

### Example B—Butanol

The reactor zone was heated to 282° C. as carbon dioxide and natural gas were supplied at 1.4 atmospheres (20 pounds per square inch) pressure with a total flow rate of 215 mL/minute. Carbon dioxide was set to 65 mL/minute while natural gas was set to 201 mL/minute. Gases were allowed to flow for 30 minutes. A colorless liquid, weight was 6.67 grams, was collected in the cold trap immersed in an ice water bath.

The liquid was dried over anhydrous sodium sulfate then filtered to collect 5.1 grams of liquid product. An FTIR spectrum of the liquid produced useful spectral information. Relatively intense absorption bands were observed at 2964, 2936 and 2873 wave numbers with relatively weaker bands at 1466, 1378, 1275, 904, 885, 798 and 753 wave numbers. In addition, a weak band was observed at 1712 wave numbers identified with ketones with an intense band at 1198 wave numbers indicating the presence of a branched alcohol. This was identified as a minor amount of acetone, as produced in the previous section, and a majority of butyl alcohol.

### Example C—Pentane

The reactor zone was heated to 280° C. as carbon dioxide and natural gas were supplied at 1.7 atmospheres (25 pounds per square inch) pressure with a total flow rate of 257 mL/minute. Carbon dioxide was set to 50 mL/minute while natural gas was set to 207 mL/minute. Gases were allowed to flow for 30 minutes. A colorless liquid, weight was 8.33 grams, was collected in the cold trap immersed in a dry ice acetone bath.

The liquid was dried over anhydrous sodium sulfate then filtered to collect 5.7 grams of cold liquid product. An FTIR spectrum of the liquid produced useful spectral information. Relatively intense absorption bands were observed at 2966, 2937 and 2875 wave numbers with relatively weaker bands at 1467, 1379, 1276, 905, 886, 798 and 754 wave numbers demonstrating a hydrocarbon liquid. In addition, a weak band at 1191 wave numbers indicating the presence of a trace of alcohol. This was identified as a low boiling hydrocarbon, pentane, and a small amount of butyl alcohol.

### Example D—Tridecane

The reactor zone was heated to 286° C. as carbon dioxide and propane gas were supplied at 1.4 atmospheres (20 pounds per square inch) pressure with a total flow rate of 257 mL/minute. Carbon dioxide was set to 50 mL/minute while propane gas was set to 210 mL/minute. Gases were allowed to flow for 30 minutes. A colorless mixed liquid, weight was 11.29 grams, was collected in the cold trap immersed in an ice water bath.

The liquid was dried over anhydrous sodium sulfate then filtered to collect 8.9 grams of an oily liquid product. An FTIR spectrum of the liquid produced useful spectral information. Relatively intense absorption bands were observed at 2966, 2937 and 2875 wave numbers with relatively weaker bands at 1467, 1379, 1276, 905, 886, 798, 754 and 720 wave numbers demonstrating a hydrocarbon liquid. In addition, a weak band at 1188 wave numbers indicating the presence of a trace of alcohol. This was identified as the liquid hydrocarbons tridecane, boiling range 230° C. to 240° C., and a trace amount of butyl alcohol.

What is claimed:

1. A chemical process for reacting carbon dioxide with a gaseous hydrocarbon, comprising natural gas, methane, ethane, propane, butane or pentane, in the presence of a transition metal oxide catalyst, selected from the group comprising manganese oxide, iron oxide or cobalt oxide, forming solvents comprising acetone, butanol, pentane, pentanone, heptanol and nonane.

2. A chemical process for reacting carbon dioxide with a gaseous hydrocarbon, comprising natural gas, methane, ethane, propane, butane or pentane, in the presence of a transition metal oxide catalyst, selected from the group comprising manganese oxide, iron oxide or cobalt oxide, at temperatures of 200° C. to 460° C. and pressures of 1 atmosphere to 7 atmospheres forming solvents comprising acetone, butanol, pentane, pentanone, heptanol and nonane.

3. A chemical process for reacting carbon dioxide with a gaseous hydrocarbon, comprising natural gas, methane, ethane, propane, butane or pentane, in the presence of a transition metal oxide catalyst, selected from the group comprising manganese oxide, iron oxide or cobalt oxide, at temperatures of 200° C. to 500° C. and pressures of 1 atmosphere to 10 atmospheres forming solvents comprising acetone, butanol, pentane, pentanone, heptanol and nonane.
